(19) United States
(12) Patent Application Publication
(10) Pub. No.: US 2017/0165182 A1
(43) Pub. Date: Jun. 15, 2017

(54) AGENT AND METHOD FOR TEMPORARILY DEFORMING KERATIN-CONTAINING FIBERS

(71) Applicant: Henkel AG & Co. KGaA, Duesseldorf (DE)

(72) Inventors: Thorsten Knappe, Schenefeld (DE); Pamela Kaftan, Hamburg (DE); Maria Catalina Bermudez Agudelo, Hamburg (DE); Tim Bethge, Hamburg (DE)

(73) Assignee: Henkel AG & Co. KGaA, Duesseldorf (DE)

(21) Appl. No.: 15/363,493

(22) Filed: Nov. 29, 2016

(30) Foreign Application Priority Data
Dec. 15, 2015 (DE) 10 2015 225 207.8

(51) Int. Cl. A61K 8/81; A61K 8/31; B65D 83/14; A61K 8/04; A61K 8/34; A61Q 5/06; A61K 8/33

(52) U.S. Cl. CPC A61K 8/8158; A61Q 5/06; A61K 8/31; A61K 8/315; A61K 8/33; A61K 8/046; A61K 8/34; B65D 83/752; A61K 2800/594; A61K 2800/87

(57) ABSTRACT

Cosmetic agents and methods for temporarily deforming keratinic fibers using the cosmetic agents are provided herein. In one embodiment, the cosmetic agent includes a cosmetic preparation. The cosmetic preparation contains at least one copolymer a1) constructed at least from the following monomer units: N-tert-octylacrylamide, acrylic acid, and tert-butylaminoethyl methacrylate. The cosmetic preparation further contains at least one copolymer a2) constructed at least from the following monomer units: N-tert-butylacrylamide, acrylic acid, and ethyl acrylate. The proportion by weight of copolymers a1) and a2) in the total weight of the cosmetic preparation is about 10 to about 30% by weight.

### CROSS-REFERENCE TO RELATED APPLICATION

This application claims priority to German Patent Application No. 10 2015 225 207.8, filed Dec. 15, 2015, which is incorporated herein by reference in its entirety.

### TECHNICAL FIELD

This disclosure relates to a cosmetic composition for hair setting or for temporarily reshaping keratinic fibers, in particular human hair, wherein the composition contains a combination of two specific copolymers in high concentration.

### BACKGROUND

The temporary creation of hairstyles for a longer period of time lasting up to a number of days generally requires the use of firming active substances. Hair treatment agents that serve to temporarily shape the hair thus play an important role. Appropriate agents for temporary deformation usually contain synthetic polymers and/or waxes as firming active substance. Agents for assisting the temporary reshaping of keratin-containing fibers can be produced, by way of example, in the form of hairspray, hair wax, hair gel or hair mousse.

The most important property of an agent for temporarily deforming hair, also referred to hereinafter as a styling agent, lies in being able to provide the treated fibers with the greatest possible hold in the newly modeled form—i.e. a form impressed on the hair. Reference is also made to a strong hairstyle hold or to a high holding power of the styling agent. The hold of a hairstyle is determined fundamentally by the type and quantity of the used firming active substances, however the further constituents of the styling agent can also have an effect.

Besides a high holding power, styling agents must also satisfy a wide range of further requirements. These can be divided roughly into properties on the hair, properties of the respective formulation, for example properties of sprayed aerosols, and properties that concern the handling of the styling agent, wherein the properties on the hair are attributed particular importance. In particular, moisture resistance, low stickiness (tack), and a balanced conditioning effect can be cited. Furthermore, a styling agent should be universally usable for all hair types where possible and should be mild on the hair and skin.

In order to satisfy the different requirements, a multiplicity of synthetic polymers which are used in styling agents have been developed as firming active substances. These polymers can be divided into cationic, anionic, non-ionic and amphoteric firming polymers.

Known amphoteric polymers that are used in hair-firming products are based on the monomers N-tert-octylacrylamide, acrylic acid, and tert-butylaminoethyl methacrylate. Appropriate polymers and their use in hair-styling gels are described for example in international application WO 2011/012464 A2.

Hairsprays based on copolymers of N-tert-butylacrylamide, acrylic acid and ethyl acrylate are described, inter alia, in German patent application DE 10 2004 040 172 A1.

Not any polymer or not any polymer blend is suitable in principle for the production of hair-styling agents. This is true in particular for hairsprays, in which case the viscosity for example and thus also the spray behavior is influenced by the polymer or the quantity of the used polymer.

Furthermore, not any polymer and any polymer mixture are suitable for producing highly concentrated hair-styling agents. Besides the previously described problems with the viscosity and dosing of the agents, difficulties also occur with regard to the solubility and the storage stability, in particular of hairsprays, with rising polymer concentration.

A hairspray with high polymer content (compact hairspray) is described for example in international patent application WO 2005/012588 A2.

Although suitable polymers and polymer combinations have been developed for some time now for use in styling concentrates in the field of temporary hair deforming, the previously attained results still have room for improvements, in particular with regard to the storage stability, application, and the holding power of these agents. In particular, currently obtainable styling agents can still be improved insofar as a good combination of holding power and long-term hold (High Humidity Curl Retention) is not always sufficiently ensured.

### BRIEF SUMMARY

Cosmetic agents and methods for temporarily deforming keratinic fibers using the cosmetic agents are provided herein. In one embodiment, the cosmetic agent includes a cosmetic preparation. The cosmetic preparation contains at least one copolymer a1) constructed at least from the following monomer units: N-tert-octylacrylamide, acrylic acid, and tert-butylaminoethyl methacrylate. The cosmetic preparation further contains at least one copolymer a2) constructed at least from the following monomer units: N-tert-butylacrylamide, acrylic acid, and ethyl acrylate. The proportion by weight of copolymers a1) and a2) in the total weight of the cosmetic preparation is about 10 to about 30% by weight.

In another embodiment, a method for temporarily deforming keratinic fibers includes the step of acting on keratinic fibers by a cosmetic agent. The cosmetic agent contains at least one copolymer a1) constructed at least from the following monomer units: N-tert-octylacrylamide, acrylic acid, and tert-butylaminoethyl methacrylate. The cosmetic preparation further contains at least one copolymer a2) constructed at least from the following monomer units: N-tert-butylacrylamide, acrylic acid, and ethyl acrylate. The proportion by weight of copolymers a1) and a2) in the total weight of the cosmetic agent is about 10 to about 30% by weight. The method further includes the step of temporarily fixing the keratinic fibers into form.

### DETAILED DESCRIPTION

The following Detailed Description is merely exemplary in nature and is not intended to limit the various embodiments or the application and uses thereof. Furthermore, there is no intention to be bound by any theory presented in the preceding background or the following detailed description.

It is contemplated herein to provide further suitable polymer combinations which are characterized by good film-forming and/or firming properties and have a very high holding power, without detriment to the flexibility and good moisture resistance—in particular resistance to perspiration and water. The polymer combination should additionally be suitable for the production of concentrated compositions with high chemical and physical stability and should be easily applied.

This has been achieved in accordance with the disclosure by a combination of two specific copolymers different from one another.

The present disclosure provides:
1. A cosmetic agent for temporarily deforming keratinic fibers, comprising
a) a cosmetic preparation, containing,
   a1) at least one copolymer constructed at least from the following monomer units:
      N-tert-octylacrylamide,
      acrylic acid,
      tert-butylaminoethyl methacrylate;
   a2) at least one copolymer constructed at least from the following monomer units:
      N-tert-butylacrylamide
      acrylic acid
      ethyl acrylate,
wherein the proportion by weight of copolymers a1) and a2) in the total weight of the cosmetic preparation is about 10 to about 30% by weight.
2. The cosmetic agent according to point 1, wherein the proportion by weight of copolymers a1) and a2) in the total weight of the cosmetic preparation is about 11 to about 25% by weight and in particular about 12 to about 20% by weight.
3. The cosmetic agent according to any one of the preceding points, wherein the at least one copolymer a1), in relation to its total weight, consists to an extent of at least 90% by weight, preferably at least 95% by weight, and in particular at least 97% by weight of the monomers N-ted-octylacrylamide, acrylic acid, and tert-butylaminoethyl methacrylate.
4. The cosmetic agent according to any one of the preceding points, wherein the copolymer a1) consists to an extent of at least 90% by weight, preferably at least 95% by weight, and in particular at least 97% by weight of the monomers N-tert-octylacrylamide, acrylic acid, tert-butylaminoethyl methacrylate, methyl methacrylate, and hydroxypropyl methacrylate.
5. The cosmetic agent according to any one of the preceding points, wherein the copolymer a1) bears the INCI name Octylaccrylamide/Acrylates/Butylaminoethyl Methacrylate.
6. The cosmetic agent according to any one of the preceding points, wherein the preparation contains, in relation to its total weight, about 1.0 to about 15% by weight, preferably about 2.0 to about 14% by weight, and in particular from about 4.0 to about 12% by weight of copolymer a1).
7. The cosmetic agent according to any one of the preceding points, wherein the at least one copolymer a2), in relation to its total weight, consists to an extent of at least 90% by weight, preferably at least 95% by weight, and in particular at least 97% by weight of the monomers
   N-tert-butylacrylamide
   acrylic acid
   ethyl acrylate.
8. The cosmetic agent according to any one of the preceding points, wherein the copolymer a2) bears the INCI name Acrylates/t-Butylacrylamide Copolymer.
9. The cosmetic agent according to any one of the preceding points, wherein the preparation contains, in relation to its total weight, about 1.0 to about 15% by weight, preferably about 2.0 to about 14% by weight, and in particular from about 4.0 to about 12% by weight of copolymer a2).
10. The cosmetic agent according to any one of the preceding points, wherein the ratio by weight of copolymer a1) to copolymer a2) is from about 1:7 to about 7:1, preferably from about 1:5 to about 5:1, and in particular from about 1:3 to about 3:1.
11. The cosmetic agent according to any one of the preceding points, wherein the preparation, in relation to its total weight, contains about 30 to about 90% by weight, preferably about 40 to about 85% by weight, and in particular about 50 to about 80% by weight of ethanol.
12. The cosmetic agent according to any one of the preceding points, wherein the preparation, in relation to its total weight, contains about 0.01 to about 30% by weight, and in particular about 5.0 to about 25% by weight of water.
13. The cosmetic agent according to any one of the preceding points, wherein the preparation, in relation to its total weight, consists to an extent of at least 70% by weight, preferably at least 80% by weight, and in particular at least 90% by weight of the copolymers a1) and a2), ethanol, and water.
14. The cosmetic agent according to any one of the preceding points, wherein the preparation a) has a viscosity (Brookfield DV2T viscometer; 20° C., spindle 2, 10 rpm) of from 10 to 1000 mPas, preferably from 15 to 500 mPas, and in particular from 20 to 200 mPas.
15. The cosmetic agent according to any one of the preceding points, wherein the agent also comprises
   b) at least one propellant.
16. The cosmetic agent according to any one of the preceding points, wherein the agent also comprises
   b) at least one propellant from the group of propane, blend of propane and butane, dimethyl ether, and 1,1-difluoroethane.
17. The cosmetic agent according to any one of the preceding points, comprising, in relation to its total weight,
   a) about 30 to about 60% by weight of the cosmetic preparation
   b) about 40 to about 70% by weight of propellant.
18. A cosmetic product, comprising
   i) a cosmetic agent according to any one of points 1 to 17
   ii) a dispensing device having a spray valve.
19. The cosmetic product according to point 18, wherein the spray valve has a maximum valve opening below 0.4 mm, preferably between about 0.22 and about 0.32 mm, and in particular between about 0.25 and about 0.30 mm.
20. Use of an agent or a product according to any one of points 1 to 19 for temporarily deforming keratin-containing fibers, in particular human hair.
21. A method for temporarily deforming keratin-containing fibers, in particular human hair, in which the keratinic fibers are acted on by a cosmetic agent according to any one of points 1 to 17 and are temporarily fixed in their form.
22. The method according to point 21, wherein the keratinic fibers are acted on with a spray rate of the cosmetic preparation of from about 18 to about 30 g/min, preferably from about 22 to about 28 g/min.

It has been surprisingly found within the scope of the present disclosure that, by combining two constituents known per se, which are already used in styling products, an improved moisture resistance of styling products can be obtained. Other properties usually required of styling products, such as lasting hold, stiffness, and low stickiness, are maintained here. A good combination of properties of this type was not anticipated in the knowledge of the individual components and was surprising. It has been found by way of experimentation that a synergistic effect of the two components, i.e. an effect going beyond that of each of the individual components separately, in respect of the moisture resistance and the holding power is obtained by the combination of said two components.

The term ‘keratinic fibers’, in accordance with the disclosure, includes fur, wool, and feathers, but in particular human hair.

The essential constituents of the cosmetic composition as contemplated herein are the amphoteric copolymer a1) and the anionic acrylate copolymer a2), which is different from the copolymer a1).

A first essential constituent of cosmetic compositions as contemplated herein is the copolymer a1). With regard to the producibility, application and cosmetic effect of cosmetic agents as contemplated herein, it has proven to be advantageous when the proportion by weight of the copolymer a1) in the total weight of the cosmetic preparation a) is about 1.0 to about 15% by weight, preferably about 2.0 to about 14% by weight, and in particular from about 4.0 to about 12% by weight.

The copolymer a1) can be attributed to the monomers N-tert-octylacrylamide, acrylic acid, tert-butylaminoethyl methacrylate and optionally further monomers.

Preferred copolymers a1) preferably consist to an extent of at least 90% by weight, preferably at least 95% by weight, and in particular at least 97% by weight of the monomers N-tert-octylacrylamide, acrylic acid, tert-butylaminoethyl methacrylate. The copolymers a1) are preferably obtained with exclusive use of the monomers N-tert-octylacrylamide, acrylic acid, tert-butylaminoethyl methacrylate.

Copolymers a1) from the monomers N-tert-octylacrylamide, acrylic acid, tert-butylaminoethyl methacrylate, methyl methacrylate and hydroxypropyl methacrylate are particularly preferred. It is particularly preferred if the copolymer a1) consists to an extent of at least 90% by weight, preferably at least 95% by weight, and in particular at least 97% by weight of the monomers N-tert-octylacrylamide, acrylic acid, tert-butylaminoethyl methacrylate, methyl methacrylate and hydroxypropyl methacrylate.

The previously described copolymers a1) are sold for example under the name Amphomer® (INCI name: Octylacrylamide/Acrylates/Butylaminoethyl Methacrylate Copolymer; CAS number 70801-07-9) by the company National Starch.

The cosmetic preparations as contemplated herein contain an anionic copolymer a2) as second essential constituent.

With regard to the producibility, application and cosmetic effect of cosmetic agents as contemplated herein, it has proven to be advantageous when the proportion by weight of the copolymer a2) in the total weight of the cosmetic preparation a) is about 1.0 to about 15% by weight, preferably about 2.0 to about 14% by weight, and in particular from about 4.0 to about 12% by weight.

The copolymer a2) can be attributed to the monomers N-tert-butylacrylamide, acrylic acid, ethyl acrylate and optionally further monomers.

Preferred copolymers a2) preferably consist to an extent of at least 90% by weight, preferably at least 95% by weight, and in particular at least 97% by weight of the monomers i) N-tert-butylacrylamide, ii) acrylic acid, iii) ethyl acrylate. Particularly preferred copolymers a2) were obtained exclusively from the monomers i) N-tert-butylacrylamide, ii) acrylic acid, iii) ethyl acrylate.

The previously described copolymers a2) are sold for example under the name Ultrahold® 8 (INCI name: Acrylates/t-Butylacrylamide Copolymer; CAS number 26062-56-6) by BASF.

The copolymer a2) is used in the cosmetic agents preferably in partially neutralized or neutralized form. At least one alkanolamine is preferably used for neutralization. The alkanolamines usable as alkalizing agents as contemplated herein are preferably selected from primary amines having a C₂-C₆ alkyl parent substance carrying at least one hydroxyl group. Particularly preferred alkanolamines are selected from the group formed from 2-aminoethan-1-ol (monoethanolamine), tris(2-hydroxyethyl)-amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol. Alkanolamines that are very particularly preferred in accordance with the disclosure are selected from the group of 2-aminoethan-1-ol, 2-amino-2-methylpropan-1-ol and 2-amino-2-methyl-propan-1,3-diol. A particularly suitable neutralizing agent here has proven to be 2-amino-2-methylpropanol. 2-amino-2-methylpropanol is used in the agents as contemplated herein preferably in a quantity that does not exceed the quantity required for neutralization of the copolymer a2). The quantity of 2-amino-2-methylpropanol used in the agents as contemplated herein is preferably about 80 to 100%, particularly preferably about 90 to 100% and in particular about 95 to 100% of the quantity required for full neutralization of the copolymer a2). In a preferred embodiment the proportion by weight of 2-amino-2-methylpropanol in the total weight of the cosmetic preparation is a) about 0.1 to about 4.0% by weight, preferably about 0.5 to about 3.0% by weight, and in particular about 1.0 to about 2.0% by weight.

The proportion by weight of the copolymers a1) and a2) in the total weight of the cosmetic preparation is about 10 to about 30% by weight. Cosmetic preparations in which the proportion by weight of the copolymers a1) and a2) in the total weight of the cosmetic preparation is about 11 to about 25% by weight, and in particular about 12 to about 20% by weight, are preferred.

Besides the total proportion by weight of the copolymers a1) and a2), the ratio by weight of the copolymers a1) and a2) to one another also influences the moisture resistance, the holding power, and the further use properties of cosmetic agents as contemplated herein. Technically particularly advantageous cosmetic agents are characterized in that the ratio by weight of copolymer a1) to copolymer a2) is from about 1:7 to about 7:1, preferably from about 1:5 to about 5:1, and in particular from about 1:3 to about 3:1.

Besides the previously described copolymers a1) and copolymers a2), the cosmetic preparations can contain further active substances, auxiliaries and nourishing ingredients.

A first group of preferably used active substances are film-forming polymers. These film-forming polymers are not identical to the previously described copolymer a1) or copolymer a2). The proportion by weight of the film-forming polymer in the total weight of the cosmetic preparation is preferably about 0.1 to about 8.0% by weight, preferably about 0.5 to about 6.0% by weight, and in particular about 1.0 to about 4.0% by weight.

Non-ionic polymers are particularly preferably used as film-forming polymers. Suitable non-ionic polymers are, for example:
Vinylpyrrolidone/vinylester copolymers, as are sold for example under the trade name Luviskol® (BASF). Luviskol® VA 64 and Luviskol® VA 73, each vinylpyrrolidone/vinyl acetate copolymers, are preferred non-ionic polymers.
Cellulose ethers, such as hydroxypropyl cellulose, hydroxyethyl cellulose and methylhydroxypropyl cellulose, as are sold for example under the trade names Culminal® and Benecel® (AQUALON).
Shellac.
Polyvinylpyrrolidones, as are sold for example under the name Luviskol® (BASF).
Siloxanes. These siloxanes can be either water-soluble or water-insoluble. Both volatile and non-volatile siloxanes are suitable, wherein compounds of which the boiling point at normal pressure is above 200° C. are understood to be non-volatile siloxanes. Preferred siloxanes are polydialkyl siloxanes, such as polydimethyl siloxane, polyalkylaryl siloxanes, such as polyphenylmethyl siloxane, ethoxylated polydialkyl siloxanes, and polydialkyl siloxanes which contain amine groups and/or hydroxyl groups.
Glycosidically-substituted silicones.

Due to their cosmetic effect in combination with the copolymers a1) and a2), film-forming polymers used with preference in accordance with the disclosure are, in particular, the polyvinylpyrrolidones (INCI name: PVP) and the vinylpyrrolidone/vinyl acetate copolymers (INCI name VP/VA copolymer), wherein the proportion by weight of these polymers is preferably limited to quantities between 1.0 and 10% by weight. Particularly preferred cosmetic preparations as contemplated herein are therefore characterized in that they also contain, in relation to their total weight, between 1.0 and 10% by weight of polyvinylpyrrolidone and/or vinylpyrrolidone/vinyl acetate copolymer, preferably polyvinylpyrrolidone. Particularly preferred cosmetic preparations have a proportion by weight of the polyvinylpyrrolidone and/or vinylpyrrolidone/vinyl acetate copolymer c) in the total weight of the cosmetic preparation of from about 2.0 to about 8.5% by weight, preferably from about 3.0 to about 7.0% by weight.

To summarize, cosmetic agents that are particularly preferred in accordance with the disclosure with the copolymers a1) and a2) and also the film-forming polymer a3) contain three polymers that are different from one another.

Protein hydrolyzates and/or derivatives thereof can be used as nourishing ingredients. Protein hydrolyzates are product mixtures which are obtained by acid-catalyzed, base-catalyzed or enzyme-catalyzed degradation of proteins. The term ‘protein hydrolyzates’ is understood in accordance with the disclosure to also mean total hydrolyzates and also individual amino acids and derivatives thereof as well as mixtures of different amino acids. The molecular weight of the protein hydrolyzates usable in accordance with the disclosure lies between 75, the molecular weight for glycine, and 200,000, and the molecular weight is preferably about 75 to about 50,000, and very particularly preferably about 75 to about 20,000 daltons.

A further group of nourishing substances is constituted by the vitamins, provitamins, vitamin precursors and/or derivatives thereof. Here, vitamins, provitamins and vitamin precursors that are usually assigned to the groups A, B, C, E, F and H are preferred in accordance with the disclosure.

Further nourishing substances are glycerol, propylene glycol, panthenol, caffeine, nicotinamide, and sorbitol.

Plant extract, but also monosaccharides or oligosaccharides and/or lipids can also be used as nourishing substance.

The composition of some used cosmetic preparations a) in which the proportion by weight of the copolymers a1) and a2) in the total weight of the cosmetic preparation is about 10 to about 30% by weight, preferably about 11 to about 25% by weight, and in particular about 12 to about 20% by weight, can be deduced form the following tables (values in % by weight relate to the total weight of the cosmetic agent unless specified otherwise).

| | Formula 1 | Formula 2 | Formula 3 | Formula 4 | Formula 5 |
|---|---|---|---|---|---|
| Copolymer a1) * | 1.0 to 15 | 1.5 to 14.5 | 2.0 to 14 | 3.0 to 13 | 4.0 to 12 |
| Copolymer a2) * | 1.0 to 15 | 1.5 to 14.5 | 2.0 to 14 | 3.0 to 13 | 4.0 to 12 |
| optional additives | to 100 | to 100 | to 100 | to 100 | to 100 |

| | Formula 6 | Formula 7 | Formula 8 | Formula 9 | Formula 10 |
|---|---|---|---|---|---|
| Octylaccrylamide/Acrylates/ | 1.0 to 15 | 1.5 to 14.5 | 2.0 to 14 | 3.0 to 13 | 4.0 to 12 |
| Butylaminoethyl Meth- | | | | | |
| acrylate Copolymer (INCI) | | | | | |
| Copolymer a2) * | 1.0 to 15 | 1.5 to 14.5 | 2.0 to 14 | 3.0 to 13 | 4.0 to 12 |
| optional additives | to 100 | to 100 | to 100 | to 100 | to 100 |

| | Formula 11 | Formula 12 | Formula 13 | Formula 14 | Formula 15 |
|---|---|---|---|---|---|
| Copolymer a1) * | 1.0 to 15 | 1.5 to 14.5 | 2.0 to 14 | 3.0 to 13 | 4.0 to 12 |
| Acrylates/t-Butylacrylamide | 1.0 to 15 | 1.5 to 14.5 | 2.0 to 14 | 3.0 to 13 | 4.0 to 12 |
| Copolymer (INCI) | | | | | |
| optional additives | to 100 | to 100 | to 100 | to 100 | to 100 |

| | Formula 16 | Formula 17 | Formula 18 | Formula 19 | Formula 20 |
|---|---|---|---|---|---|
| Octylaccrylamide/Acrylates/ | 1.0 to 15 | 1.5 to 14.5 | 2.0 to 14 | 3.0 to 13 | 4.0 to 12 |
| Butylaminoethyl Meth- | | | | | |
| acrylate Copolymer (INCI) | | | | | |
| Acrylates/t-Butylacrylamide | 1.0 to 15 | 1.5 to 14.5 | 2.0 to 14 | 3.0 to 13 | 4.0 to 12 |
| Copolymer (INCI) | | | | | |
| optional additives | to 100 | to 100 | to 100 | to 100 | to 100 |

Preferred cosmetic agents are based on an aqueous, aqueous/alcoholic or alcoholic carrier. Preferred cosmetic agents thus contain, in relation to their total weight, about 40 to about 98% by weight, preferably about 60 to about 95% by weight, and in particular about 70 to about 92% by weight of polar solvent, preferably polar solvent from the group of water, ethanol and isopropanol.

As already mentioned, the lower alcohols conventionally used for cosmetic purposes having 1 to 4 carbon atoms, such as ethanol and isopropanol, can be contained as alcohols, in particular.

Besides these alcoholic solvents, water-soluble co-solvents can also be used in addition, in particular in combination with water. Examples of particularly preferred co-solvents are glycerol and/or ethylene glycol and/or 1,2 propylene glycol, which are preferably used in a quantity of from 0 to about 30% by weight in relation to the cosmetic preparation a).

Together with the copolymers a1) and a2) described further above, the aqueous, aqueous/alcoholic or alcoholic carriers preferably form an essential constituent of cosmetic preparations a) as contemplated herein. Cosmetic preparations which, in relation to their total weight, consist to an extent of at least 70% by weight, preferably at least 80% by weight, and in particular at least 90% by weight of copolymers a) and a2), ethanol and/or water are particularly preferred.

The composition of some technically advantageous, cosmetic preparations a) with liquid carrier, in which the proportion by weight of the copolymers a1) and a2) in the total weight of the cosmetic preparation is about 10 to about 30% by weight, preferably about 11 to about 25% by weight, and in particular about 12 to about 20% by weight can be deduced from the following tables (the values in % by weight relate to the total weight of the cosmetic agent unless specified otherwise.)

| | Formula 21 | Formula 22 | Formula 23 | Formula 24 | Formula 25 |
|---|---|---|---|---|---|
| Copolymer a1) * | 1.0 to 15 | 1.5 to 14.5 | 2.0 to 14 | 3.0 to 13 | 4.0 to 12 |
| Copolymer a2) * | 1.0 to 15 | 1.5 to 14.5 | 2.0 to 14 | 3.0 to 13 | 4.0 to 12 |
| Water and/or ethanol | 40 to 98 | 40 to 98 | 60 to 95 | 60 to 95 | 70 to 92 |
| optional additives | to 100 | to 100 | to 100 | to 100 | to 100 |

| | Formula 26 | Formula 27 | Formula 28 | Formula 29 | Formula 30 |
|---|---|---|---|---|---|
| Octylaccrylamide/Acrylates/ | 1.0 to 15 | 1.5 to 14.5 | 2.0 to 14 | 3.0 to 13 | 4.0 to 12 |
| Butylaminoethyl Meth- | | | | | |
| acrylate Copolymer (INCI) | | | | | |
| Copolymer a2) * | 1.0 to 15 | 1.5 to 14.5 | 2.0 to 14 | 3.0 to 13 | 4.0 to 12 |
| Water and/or ethanol | 40 to 98 | 40 to 98 | 60 to 95 | 60 to 95 | 70 to 92 |
| optional additives | to 100 | to 100 | to 100 | to 100 | to 100 |

| | Formula 31 | Formula 32 | Formula 33 | Formula 34 | Formula 35 |
|---|---|---|---|---|---|
| Copolymer a1) * | 1.0 to 15 | 1.5 to 14.5 | 2.0 to 14 | 3.0 to 13 | 4.0 to 12 |
| Acrylates/t-Butylacrylamide | 1.0 to 15 | 1.5 to 14.5 | 2.0 to 14 | 3.0 to 13 | 4.0 to 12 |
| Copolymer (INCI) | | | | | |
| Water and/or ethanol | 40 to 98 | 40 to 98 | 60 to 95 | 60 to 95 | 70 to 92 |
| optional additives | to 100 | to 100 | to 100 | to 100 | to 100 |

| | Formula 36 | Formula 37 | Formula 38 | Formula 39 | Formula 40 |
|---|---|---|---|---|---|
| Octylaccrylamide/Acrylates/ | 1.0 to 15 | 1.5 to 14.5 | 2.0 to 14 | 3.0 to 13 | 4.0 to 12 |
| Butylaminoethyl Meth- | | | | | |
| acrylate Copolymer (INCI) | | | | | |
| Acrylates/t-Butylacrylamide | 1.0 to 15 | 1.5 to 14.5 | 2.0 to 14 | 3.0 to 13 | 4.0 to 12 |
| Copolymer (INCI) | | | | | |
| Water and/or ethanol | 40 to 98 | 40 to 98 | 60 to 95 | 60 to 95 | 70 to 92 |
| optional additives | to 100 | to 100 | to 100 | to 100 | to 100 |

* according to claim 1

Of course, not only can the proportion by weight of the liquid carrier in the total weight of the cosmetic preparation a) vary, but the ratio by weight of aqueous to alcoholic carrier is also variable.

Preferred cosmetic preparations contain, in relation to their total weight, about 0.01 to about 30% by weight and in particular about 5.0 to about 25% by weight water.

As already mentioned, the lower alcohols conventionally used for cosmetic purposes having 1 to 4 carbon atoms, such as ethanol and isopropanol, can be contained as alcohols, in particular. Preferred cosmetic preparations contain, in relation to their total weight, about 30 to about 90, preferably about 40 to about 85% by weight, and in particular about 50 to about 80% by weight ethanol.

The composition of some technically advantageous cosmetic preparations a) with liquid carrier, in which the proportion by weight of copolymers a1) and a2) in the total weight of the cosmetic preparation is about 10 to about 30% by weight, preferably about 11 to about 25% by weight, and in particular about 12 to about 20% by weight, can be deduced from the following tables. (The values in % by weight relate to the total weight of the cosmetic agent, unless specified otherwise.)

| | Formula 41 | Formula 42 | Formula 43 | Formula 44 | Formula 45 |
|---|---|---|---|---|---|
| Copolymer a1) * | 1.0 to 15 | 1.5 to 14.5 | 2.0 to 14 | 3.0 to 13 | 4.0 to 12 |
| Copolymer a2) * | 1.0 to 15 | 1.5 to 14.5 | 2.0 to 14 | 3.0 to 13 | 4.0 to 12 |
| Ethanol | 30 to 90 | 30 to 90 | 40 to 85 | 40 to 85 | 50 to 80 |
| Water | 0.01 to 30 | 0.1 to 30 | 1.0 to 30 | 2.0 to 30 | 5.0 to 25 |
| optional additives | to 100 | to 100 | to 100 | to 100 | to 100 |

| | Formula 46 | Formula 47 | Formula 48 | Formula 49 | Formula 50 |
|---|---|---|---|---|---|
| Octylaccrylamide/Acrylates/ | 1.0 to 15 | 1.5 to 14.5 | 2.0 to 14 | 3.0 to 13 | 4.0 to 12 |
| Butylaminoethyl Meth- | | | | | |
| acrylate Copolymer (INCI) | | | | | |
| Copolymer a2) * | 1.0 to 15 | 1.5 to 14.5 | 2.0 to 14 | 3.0 to 13 | 4.0 to 12 |
| Ethanol | 30 to 90 | 30 to 90 | 40 to 85 | 40 to 85 | 50 to 80 |
| Water | 0.01 to 30 | 0.1 to 30 | 1.0 to 30 | 2.0 to 30 | 5.0 to 25 |
| optional additives | to 100 | to 100 | to 100 | to 100 | to 100 |

| | Formula 51 | Formula 52 | Formula 53 | Formula 54 | Formula 55 |
|---|---|---|---|---|---|
| Copolymer a1) * | 1.0 to 15 | 1.5 to 14.5 | 2.0 to 14 | 3.0 to 13 | 4.0 to 12 |
| Acrylates/t-Butylacrylamide | 1.0 to 15 | 1.5 to 14.5 | 2.0 to 14 | 3.0 to 13 | 4.0 to 12 |
| Copolymer (INCI) | | | | | |
| Ethanol | 30 to 90 | 30 to 90 | 40 to 85 | 40 to 85 | 50 to 80 |
| Water | 0.01 to 30 | 0.1 to 30 | 1.0 to 30 | 2.0 to 30 | 5.0 to 25 |
| optional additives | to 100 | to 100 | to 100 | to 100 | to 100 |

| | Formula 56 | Formula 57 | Formula 58 | Formula 59 | Formula 60 |
|---|---|---|---|---|---|
| Octylaccrylamide/Acrylates/ | 1.0 to 15 | 1.5 to 14.5 | 2.0 to 14 | 3.0 to 13 | 4.0 to 12 |
| Butylaminoethyl Meth- | | | | | |
| acrylate Copolymer (INCI) | | | | | |
| Acrylates/t-Butylacrylamide | 1.0 to 15 | 1.5 to 14.5 | 2.0 to 14 | 3.0 to 13 | 4.0 to 12 |
| Copolymer (INCI) | | | | | |
| Ethanol | 30 to 90 | 30 to 90 | 40 to 85 | 40 to 85 | 50 to 80 |
| Water | 0.01 to 30 | 0.1 to 30 | 1.0 to 30 | 2.0 to 30 | 5.0 to 25 |
| optional additives | to 100 | to 100 | to 100 | to 100 | to 100 |

* according to claim 1

The cosmetic preparation is preferably sprayed onto the hair. This is performed particularly preferably with use of a propellant (aerosol spray). Preferred cosmetic agents therefore also comprise at least one propellant b) in addition to the cosmetic preparation a).

Suitable propellants (propellant gases) are propane, propene, n-butane, iso-butane, iso-butene, n-pentane, pentene, iso-pentane, iso-pentene, methane, ethane, dimethyl ether, nitrogen, air, oxygen, nitrous oxide, 1,1,1,3-tetrafluorethane, heptafluoro-n-propane, perfluoroethane, monochlorodifluoromethane, 1,1-difluoroethane, more specifically either individually or in combination. Hydrophilic propellant gases, such as carbon dioxide, can also be used advantageously within the sense of the present disclosure if the proportion of hydrophilic gases is selected to be low and lipophilic propellant gas (for example propane/butane) is present in excess. Propane, n-butane, iso-butane and mixtures of these propellant gases are particularly preferred. Preferred cosmetic agents are characterized in that the agent also comprises at least one propellant b) from the group of propane, mixture of propane and butane, dimethyl ether and 1,1-difluoroethane (INCI: Hydrofluorocarbon 152a).

Preferred cosmetic agents as contemplated herein contain, in relation to their total weight, about 30 to about 60% by weight of the cosmetic preparation a) and also about 40 to about 70% by weight of propellant b).

The further composition of some preferred cosmetic agents which also comprise a propellant b) in addition to the cosmetic preparation a) and in which the proportion by weight of the copolymers a1) and a2) in the total weight of the cosmetic preparation is about 10 to about 30% by weight, preferably about 11 to about 25% by weight, and in particular about 12 to about 20% by weight, can be deduced from the following table 1.

In table 1, the left-hand column (“Formula x”) refers to one of the exemplary cosmetic preparations a) of formulas 1 to 60 specified in the tables presented further above. The other columns two to five (“Propellant”) each specify the quantity of propellant combined with the corresponding cosmetic preparation. These values in “% by weight” relate to the total weight of the cosmetic preparation a) of the respective “Formula x” without propellant.

The value “50 to 200% by weight” in the following table 1 corresponds to the addition of propellant to the cosmetic preparation a) in a quantity of from about 50 to about 200% by weight of the weight of the cosmetic preparation a). In other words, the cosmetic preparation a) and the propellant b) are present in this cosmetic agent in a ratio by weight of from about 100:50 to about 100:200 or from about 2:1 to about 1:2.

The cosmetic agents according to row 4, column 4 in the following table 1 are therefore a mixture of the propellant-free cosmetic preparation a) according to formula 3 with a propane/butane mixture in a ratio by weight of cosmetic preparation a) to propellant of from about 100:50 to about 100:200. The entry in row 4, column 4 in other words describes a cosmetic agent for temporarily deforming keratinic fibers, comprising
a) a cosmetic preparation, containing,
   a1) about 2.0 to about 14% by weight of at least one copolymer constructed at least from the following monomer units:
      N-tert-octylacrylamide,
      acrylic acid,
      tert-butylaminoethyl methacrylate;
   a2) about 2.0 to about 14% by weight of at least one copolymer constructed at least from the following monomer units:
      N-tert-butylacrylamide
      acrylic acid
      ethyl acrylate,
b) propellant from the group propane/butane mixtures,
   wherein the proportion by weight of copolymers a1) and a2) in the total weight of the cosmetic preparation is about 10 to about 30% by weight and the ratio by weight of cosmetic preparation a) to propellant b) is about 2:1 to about 1:2.

TABLE 1

| | Propellant [% by weight] | | | |
|---|---|---|---|---|
| Formula 1 | 50 to 200 | 50 to 200 DFE* | 50 to 200 P/B** | 50 to 200 DME*** |
| Formula 2 | 50 to 200 | 50 to 200 DFE* | 50 to 200 P/B** | 50 to 200 DME*** |
| Formula 3 | 50 to 200 | 50 to 200 DFE* | 50 to 200 P/B** | 50 to 200 DME*** |
| Formula 4 | 50 to 200 | 50 to 200 DFE* | 50 to 200 P/B** | 50 to 200 DME*** |
| Formula 5 | 50 to 200 | 50 to 200 DFE* | 50 to 200 P/B** | 50 to 200 DME*** |
| Formula 6 | 50 to 200 | 50 to 200 DFE* | 50 to 200 P/B** | 50 to 200 DME*** |
| Formula 7 | 50 to 200 | 50 to 200 DFE* | 50 to 200 P/B** | 50 to 200 DME*** |
| Formula 8 | 50 to 200 | 50 to 200 DFE* | 50 to 200 P/B** | 50 to 200 DME*** |
| Formula 9 | 50 to 200 | 50 to 200 DFE* | 50 to 200 P/B** | 50 to 200 DME*** |
| Formula 10 | 50 to 200 | 50 to 200 DFE* | 50 to 200 P/B** | 50 to 200 DME*** |
| Formula 11 | 50 to 200 | 50 to 200 DFE* | 50 to 200 P/B** | 50 to 200 DME*** |
| Formula 12 | 50 to 200 | 50 to 200 DFE* | 50 to 200 P/B** | 50 to 200 DME*** |
| Formula 13 | 50 to 200 | 50 to 200 DFE* | 50 to 200 P/B** | 50 to 200 DME*** |
| Formula 14 | 50 to 200 | 50 to 200 DFE* | 50 to 200 P/B** | 50 to 200 DME*** |
| Formula 15 | 50 to 200 | 50 to 200 DFE* | 50 to 200 P/B** | 50 to 200 DME*** |
| Formula 16 | 50 to 200 | 50 to 200 DFE* | 50 to 200 P/B** | 50 to 200 DME*** |
| Formula 17 | 50 to 200 | 50 to 200 DFE* | 50 to 200 P/B** | 50 to 200 DME*** |
| Formula 18 | 50 to 200 | 50 to 200 DFE* | 50 to 200 P/B** | 50 to 200 DME*** |
| Formula 19 | 50 to 200 | 50 to 200 DFE* | 50 to 200 P/B** | 50 to 200 DME*** |
| Formula 20 | 50 to 200 | 50 to 200 DFE* | 50 to 200 P/B** | 50 to 200 DME*** |
| Formula 21 | 50 to 200 | 50 to 200 DFE* | 50 to 200 P/B** | 50 to 200 DME*** |
| Formula 22 | 50 to 200 | 50 to 200 DFE* | 50 to 200 P/B** | 50 to 200 DME*** |
| Formula 23 | 50 to 200 | 50 to 200 DFE* | 50 to 200 P/B** | 50 to 200 DME*** |
| Formula 24 | 50 to 200 | 50 to 200 DFE* | 50 to 200 P/B** | 50 to 200 DME*** |
| Formula 25 | 50 to 200 | 50 to 200 DFE* | 50 to 200 P/B** | 50 to 200 DME*** |
| Formula 26 | 50 to 200 | 50 to 200 DFE* | 50 to 200 P/B** | 50 to 200 DME*** |
| Formula 27 | 50 to 200 | 50 to 200 DFE* | 50 to 200 P/B** | 50 to 200 DME*** |
| Formula 28 | 50 to 200 | 50 to 200 DFE* | 50 to 200 P/B** | 50 to 200 DME*** |
| Formula 29 | 50 to 200 | 50 to 200 DFE* | 50 to 200 P/B** | 50 to 200 DME*** |
| Formula 30 | 50 to 200 | 50 to 200 DFE* | 50 to 200 P/B** | 50 to 200 DME*** |
| Formula 31 | 50 to 200 | 50 to 200 DFE* | 50 to 200 P/B** | 50 to 200 DME*** |
| Formula 32 | 50 to 200 | 50 to 200 DFE* | 50 to 200 P/B** | 50 to 200 DME*** |
| Formula 33 | 50 to 200 | 50 to 200 DFE* | 50 to 200 P/B** | 50 to 200 DME*** |
| Formula 34 | 50 to 200 | 50 to 200 DFE* | 50 to 200 P/B** | 50 to 200 DME*** |
| Formula 35 | 50 to 200 | 50 to 200 DFE* | 50 to 200 P/B** | 50 to 200 DME*** |
| Formula 36 | 50 to 200 | 50 to 200 DFE* | 50 to 200 P/B** | 50 to 200 DME*** |
| Formula 37 | 50 to 200 | 50 to 200 DFE* | 50 to 200 P/B** | 50 to 200 DME*** |
| Formula 38 | 50 to 200 | 50 to 200 DFE* | 50 to 200 P/B** | 50 to 200 DME*** |
| Formula 39 | 50 to 200 | 50 to 200 DFE* | 50 to 200 P/B** | 50 to 200 DME*** |
| Formula 40 | 50 to 200 | 50 to 200 DFE* | 50 to 200 P/B** | 50 to 200 DME*** |
| Formula 41 | 50 to 200 | 50 to 200 DFE* | 50 to 200 P/B** | 50 to 200 DME*** |
| Formula 42 | 50 to 200 | 50 to 200 DFE* | 50 to 200 P/B** | 50 to 200 DME*** |
| Formula 43 | 50 to 200 | 50 to 200 DFE* | 50 to 200 P/B** | 50 to 200 DME*** |
| Formula 44 | 50 to 200 | 50 to 200 DFE* | 50 to 200 P/B** | 50 to 200 DME*** |
| Formula 45 | 50 to 200 | 50 to 200 DFE* | 50 to 200 P/B** | 50 to 200 DME*** |
| Formula 46 | 50 to 200 | 50 to 200 DFE* | 50 to 200 P/B** | 50 to 200 DME*** |
| Formula 47 | 50 to 200 | 50 to 200 DFE* | 50 to 200 P/B** | 50 to 200 DME*** |
| Formula 48 | 50 to 200 | 50 to 200 DFE* | 50 to 200 P/B** | 50 to 200 DME*** |
| Formula 49 | 50 to 200 | 50 to 200 DFE* | 50 to 200 P/B** | 50 to 200 DME*** |
| Formula 50 | 50 to 200 | 50 to 200 DFE* | 50 to 200 P/B** | 50 to 200 DME*** |
| Formula 51 | 50 to 200 | 50 to 200 DFE* | 50 to 200 P/B** | 50 to 200 DME*** |
| Formula 52 | 50 to 200 | 50 to 200 DFE* | 50 to 200 P/B** | 50 to 200 DME*** |
| Formula 53 | 50 to 200 | 50 to 200 DFE* | 50 to 200 P/B** | 50 to 200 DME*** |
| Formula 54 | 50 to 200 | 50 to 200 DFE* | 50 to 200 P/B** | 50 to 200 DME*** |
| Formula 55 | 50 to 200 | 50 to 200 DFE* | 50 to 200 P/B** | 50 to 200 DME*** |
| Formula 56 | 50 to 200 | 50 to 200 DFE* | 50 to 200 P/B** | 50 to 200 DME*** |
| Formula 57 | 50 to 200 | 50 to 200 DFE* | 50 to 200 P/B** | 50 to 200 DME*** |
| Formula 58 | 50 to 200 | 50 to 200 DFE* | 50 to 200 P/B** | 50 to 200 DME*** |
| Formula 59 | 50 to 200 | 50 to 200 DFE* | 50 to 200 P/B** | 50 to 200 DME*** |
| Formula 60 | 50 to 200 | 50 to 200 DFE* | 50 to 200 P/B** | 50 to 200 DME*** |

*“DFE” corresponds to 1,1-difluoroethane
**“P/B” corresponds to a propane/butane mixture
***“DME” corresponds to dimethyl ether

Vessels made of metal (aluminum, tinplate, tin), protected plastic or non-splintering plastic, or glass coated externally with plastic are potential compressed-gas containers for aerosol applications, wherein the compressive strength, breaking strength, corrosion resistance, ease of filling and also aesthetic considerations, ease of handling, printability, etc. play a role when selecting such a vessel. Special internal protective coatings ensure resistance to corrosion in respect of the cosmetic agent a).

If the agents as contemplated herein are sprayed onto the hair, these agents are advantageously provided with a dispensing device and a spray valve. The resulting cosmetic products accordingly comprise a cosmetic agent as contemplated herein and also a dispensing device having a spray valve. Spray valves which have a maximum valve opening less than 0.4 mm, preferably between about 0.22 and about 0.32 mm, and in particular between about 0.25 and about 0.30 mm are preferred. The valve opening area is preferably about 60×10⁻³ to about 100×10⁻³ mm².

The spray rate in methods as contemplated herein is preferably from about 18 to about 30 g/min, in particular from about 22 to about 28 g/min.

Besides being determined by the pressure within the compressed-gas container and the valve opening, the spray rate is also additionally determined by the viscosity of the cosmetic preparation a). Preferred cosmetic agents are characterized in that the preparation a) has a viscosity (Brookfield DV2T viscometer; 20° C., spindle 2, 10 rpm) of from about 10 to about 1000 mPas, preferably from about 15 to about 500 mPas, and in particular from about 20 to about 200 mPas.

In a preferred embodiment of the disclosure the valve has a valve cone coated with a paint or a polymer plastic A and a flexible element with recovery characteristics, which returns the valve after actuation into the closed position (=rest position of the valve). Corresponding cosmetic products in which the aerosol dispensing device comprises a valve which has a valve cone and/or a flexible element with recovery characteristics which is/are coated by a paint or a polymer plastic A are preferred in accordance with the disclosure.

In a further preferred embodiment of the disclosure the valve has a flexible element with recovery characteristics and/or a valve cone formed from at least one plastic B, preferably an elastomer plastic. Here too, cosmetic products as contemplated herein in which the valve has a flexible element with recovery characteristics and/or a valve cone formed from at least one plastic B are preferred, wherein preferred plastics B are elastomer plastics. Particularly preferred elastomer plastics are selected from Buna, in particular Buna N, Buna 421, Buna 1602 and Buna KA 6712, neoprene, butyl and chlorobutyl.

In a further preferred embodiment of the disclosure the flexible element with recovery characteristics can be formed as a coil spring or coil compression spring. In a further preferred embodiment of the disclosure the flexible element with recovery characteristics can be formed in one piece with the valve cone and can have flexible limbs.

As mentioned in the introduction, the previously described cosmetic agents are characterized by particular hair-related cosmetic properties, in particular advantageous properties in respect of temporary hair deformation. A second subject of the present disclosure is therefore the use of an agent as contemplated herein for temporarily deforming keratin-containing fibers, in particular human hair.

A third subject of the present application is a method for temporarily deforming keratin-containing fibers, in particular human hair, in which the keratinic fibers are acted on by a cosmetic agent as contemplated herein and are temporarily fixed in their form.

While at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing an exemplary embodiment of the disclosure. It being understood that various changes may be made in the function and arrangement of elements described in an exemplary embodiment without departing from the scope of the disclosure as set forth in the appended claims.

1. A cosmetic agent for temporarily deforming keratinic fibers, comprising:
a cosmetic preparation, containing:
at least one copolymer a1) constructed at least from the following monomer units;
N-tert-octylacrylamide,
acrylic acid, and
tert-butylaminoethyl methacrylate; and
at least one copolymer a2) constructed at least from the following monomer units;
N-tert-butylacrylamide,
acrylic acid, and
ethyl acrylate;
wherein the proportion by weight of copolymers a1) and a2) in the total weight of the cosmetic preparation is about 10 to about 30% by weight.

2. The cosmetic agent according to claim 1, wherein the proportion by weight of copolymers a1) and a2) in the total weight of the cosmetic preparation is about 11 to about 25% by weight.

3. The cosmetic agent according to claim 1, wherein the preparation, in relation to its total weight, contains about 1.0 to about 15% by weight of copolymer a1).

4. The cosmetic agent according to claim 1, wherein the preparation, in relation to its total weight, contains about 1.0 to about 15% by weight of copolymer a2).

5. The cosmetic agent according to claim 1, wherein the preparation, in relation to its total weight, contains about 30 to about 90% by weight of ethanol.

6. The cosmetic agent according to claim 1, wherein the preparation, in relation to its total weight, contains about 0.01 to about 30% by weight of water.

7. The cosmetic agent according to claim 1, wherein the agent also comprises at least one propellant.

8. A cosmetic product, comprising:
a cosmetic agent according to claim 1, and
a dispensing device having a spray valve.

9. (canceled)

10. (canceled)

11. The cosmetic agent according to claim 1, wherein the proportion by weight of copolymers a1) and a2) in the total weight of the cosmetic preparation is about 12 to about 20% by weight.

12. The cosmetic agent according to claim 1, wherein the preparation, in relation to its total weight, contains about 2.0 to about 14% by weight of copolymer a1).

13. The cosmetic agent according to claim 1, wherein the preparation, in relation to its total weight, contains about 4.0 to about 12% by weight of copolymer a1).

14. The cosmetic agent according to claim 1, wherein the preparation, in relation to its total weight, contains about 2.0 to about 14% by weight of copolymer a2).

15. The cosmetic agent according to claim 1, wherein the preparation, in relation to its total weight, contains about 4.0 to about 12% by weight of copolymer a2).

16. The cosmetic agent according to claim 1, wherein the preparation, in relation to its total weight, contains about 40 to about 85% by weight of ethanol.

17. The cosmetic agent according to claim 1, wherein the preparation, in relation to its total weight, contains about 50 to about 85% by weight of ethanol.

18. The cosmetic agent according to claim 1, wherein the preparation, in relation to its total weight, contains about 5 to about 25% by weight of water.

19. The cosmetic agent according to claim 1, wherein the at least one copolymer a1), in relation to its total weight, consists to an extent of at least 90% by weight of the monomers N-tert-octylacrylamide, acrylic acid, tert-butylaminoethyl methacrylate, methyl methacrylate, and hydroxypropyl methacrylate.

20. The cosmetic agent according to claim 1, wherein the at least one copolymer a2), in relation to its total weight, consists to an extent of at least 90% by weight of the monomers N-tert-butylacrylamide, acrylic acid, and ethyl acrylate.

21. A cosmetic agent for temporarily deforming keratinic fibers, comprising:
a cosmetic preparation, containing:
at least one copolymer a1) constructed at least from the following monomer units;
N-tert-octylacrylamide,
acrylic acid, and
tert-butylaminoethyl methacrylate;
at least one copolymer a2) constructed at least from the following monomer units;
N-tert-butylacrylamide,
acrylic acid, and
ethyl acrylate;
at least one propellant from the group propane, mixture of propane and butane, dimethyl ether and 1,1-difluoroethane; and
at least one of ethanol or water;
wherein the proportion by weight of copolymers a1) and a2) in the total weight of the cosmetic preparation is about 10 to about 30% by weight.

22. A method for temporarily deforming keratinic fibers, the method comprising the steps of:
acting on keratinic fibers by a cosmetic agent comprising:
at least one copolymer a1) constructed at least from the following monomer units;
N-tert-octylacrylamide,
acrylic acid, and
tert-butylaminoethyl methacrylate; and
at least one copolymer a2) constructed at least from the following monomer units;
N-tert-butylacrylamide,
acrylic acid, and
ethyl acrylate;
wherein the proportion by weight of copolymers a1) and a2) in the total weight of the cosmetic agent is about 10 to about 30% by weight; and
temporarily fixing the keratinic fibers into form.
